(19) United States
(12) Patent Application Publication
(10) Pub. No.: US 2009/0148504 A1
(43) Pub. Date: Jun. 11, 2009

(54) TRANSDERMAL PHARMACEUTICAL PREPARATION

(76) Inventors: Fumio Kamiyama, Kyoto-city (JP); Ying-shu Quan, Kyoto-city (JP)

Correspondence Address: Cheng Law Group, PLLC, 1100 17th Street, N.W., Suite 503, Washington, DC, 20036, US

(21) Appl. No.: 12/359,073

(22) Filed: Jan. 23, 2009

(30) Foreign Application Priority Data
Jul. 25, 2007 (JP) 2007-214145

(51) Int. Cl. A61F 13/02; A61K 31/4468

(52) U.S. Cl. 424/448; 514/329

(57) ABSTRACT

A percutaneous administration type pharmaceutical preparation in tape form comprising a flexible backing, which is not permeable by the active ingredient, and an adhesive base layer formed on said flexible backing; said adhesive base layer consisting essentially of an adhesive base material, ingredients, and an active ingredient, wherein said adhesive base is a copolymer containing one or more (meth) alkyl acrylate monomers and one or more monomers copolymerizable with the (a) monomer selected from the group of unsaturated ethylenically unsaturated monomers, and said ingredients are diethylene glycol monocetyl ether, octanoic acid and/or oleic acid, lauric acid diethanolamide, polyvinylpyrollidone; and said active ingredient is fentanyl.

### BACKGROUND OF THE INVENTION

1. Field of invention

The present invention relates to a transdermal administration type pharmaceutical preparation in tape form containing fentanyl as an active ingredient therein.

2. Description of the Prior Art

Fentanyl is known as an extremely potent narcotic analgesic and could be used as transdermal delivery. For example, the transdermal fentanyl reservoir patch with fentanyl dispersed in liquid or gel state in reservoir has been on market. However, fentanyl will touch the skin in large area and result in overdose delivery once fentanyl leak out from the drug reservoir. An overdose fentanyl will cause quick breath inhibition and can be fatal. In order to overcome the deficiency, a transdermal administration type pharmaceutical preparation in tape form has been proposed. For example, the tape form comprises of a flexible backing, which is not permeable by the active ingredient, and an adhesive base layer as well as a releasing liner; said adhesive base layer consisting of acrylic adhesive base material and fentanyl or its analgetically effective derivatives; wherein polyacrylic adhesive characterized by self-adhesive and no carboxyl group, and the saturated solubility of fentanyl in it should be 3 to 20 weight %, preferably 4% to 14% by weight, most preferably 5% to 10% by weight. Therefore, the adhesive base layer of said tape form contain at least 80 weight % active ingredient. (See Potent 1, JP2005-501111) However, the above tape could not supply fentanyl transdermally at a constant rate over a prolonged period of time.

### DESCRIPTION OF THE INVENTION

An object of this invention is to provide a transdermal administration type pharmaceutical preparation in tape form which has better adhesive force and appropriate skin adhesion, and could supply fentanyl gradually and at a constant rate over a prolonged period of application.

The transdermal administration type pharmaceutical preparation in tape form of this invention comprises of a flexible backing which is not permeable by the active ingredient, and an adhesive base layer formed on said flexible backing; said adhesive base layer consisting essentially of an adhesive base material, ingredients and an active ingredient compatible with said adhesive base material. The said adhesive base material is a copolymer one or more (meth) alkyl acrylate monomers which has 4 to 10 carbon atoms in the alkyl group, and one or more monomers copolymerizable with the (meth)alkyl acrylate monomer selected from the group of unsaturated ethylenically unsaturated monomers.

The ingredients are the mixture of diethylene glycol monocetyl ether, octanoic acid and/or oleic acid, lauric acid diethanolamide, and polyvinylpyrollidone. The active ingredient is fentanyl.

The said adhesive base layer is the homogenous mixture of adhesive, ethylene glycol monocetyl ether, octanoic acid and/or oleic acid, lauric acid diethanolamide, polyvinylpolypyrrolidone and fentanyl in a concentration ratio of 100:1 to 10:1 to 10:1 to 5:5 to 20:5 to 15 by weight respectively.

The flexible backing used in this invention is not permeable by fentanyl, examples of which are a single film composed of a cellulose acetate, a ethyl cellulose, a polyethylene resin, a polypropylene resin, a ethylene-propylene copolymer, a ethylene-vinyl acetate copolymer, a polyvinyl chloride resin, a polyvinyllidene chloride resin, a vinyl chloride-vinyl acetate copolymer, a polyamide resin, a polyester resin, a ABS resin, a SIS resin, a SEBS resin, a polyurethane resin, a silicone resin, or an aluminum film; a laminated film composed of these single films. Besides, the said flexible backing could be a single woven or non woven; or a laminated woven or non woven. The thickness of the said flexible backing in this invention is 10 to 1000 μm. It is more preferably in the range of 2 to 100 μm.

The transdermal administration type pharmaceutical preparation in tape form of this invention comprises of adhesive base material, diethylene glycol monocetyl ether, octanoic acid and/or oleic acid, lauric acid diethanolamide, polyvinylpolypyrrolidone and fentanyl in a concentration ratio of 100:1 to 10:1 to 10:1 to 5:5 to 20:5 to 15 by weight respectively.

The said adhesive base material in this invention is a copolymer of monomer A which is one or more monomers selected from the group consisting (methyl) alkyl acrylate and monomer B which is one or more monomers selected from unsaturated vinyl group compatible with monomer A. Examples of monomer A are butyl acrylate, octyl 2-propenoate, 2-ethylhexyl acrylate, or 2-ethylhexyl methacrylate etc. It is more preferably to select 2-ethylhexyl acrylate. Examples of monomer B are acrylic acid, methacrylic acid, vinyl acetate, styrene, vynylpyrollidone, etc. More specifically, the copolymer contains monomer A in a concentration of 60 to 90% by weight of the total amount of monomers, preferably 70 to 85%, and monomer B in a concentration of 40 to 10% by weight of the total amount of monomers, preferably 30 to 15%. If monomer A is less than 60 weight %, the adhesiveness of the adhesive base material will be reduced. If monomer A is larger than 90%, the cohesive force of the adhesive base material will be reduced, making it difficult for the patch to be peeled off the skin.

The said adhesive base material can be an acrylic adhesive available on the market, such as DURO-TAC 87-2051, DURO-TAC 87-2677 (National Starch, USA).

The said adhesive base material can be crosslinked. Examples of crosslinking agents are chemical crosslinking agents such as polyisocyanate etc., or ionic crosslinking agent such as aluminum acetylacetonate. DURO-TAC 87-2677 is a self crosslinkable adhesive. There is no necessary to add crosslinking agent to this adhesive for the crosslinking.

The said adhesive base material can be polymerized by any known method, such as solution polymerization, emulsion polymerization, suspension polymerization, or bulk polymerization etc.

The said ingredient of diethylene glycol monocetyl ether in this invention is a chemical transdermal enhancer. If it is added less than necessary, the enhancing effect on fentanyl transdermal delivery will be reduced; if it is added larger than necessary, the cohesive force of the adhesive base material will be reduced. Thus, the adhesive base material and ethylene glycol monocetyl ether are mixed with the adhesive base material/diethylene glycol monocetyl ether ratio by weight in the range from 100/1 to 100/10.

The said ingredient of octanoic acid and/or oleic acid in this invention is not only a chemical transdermal enhancer, but also is used to prevent crystallization of fentanyl from the adhesive base layer, therefore increasing the solubility of fentanyl in adhesive base material. However, a larger addition of octanoic acid and/or oleic acid than necessary will result in softening of the adhesive base material which makes it easier for the adhesive to remain on the skin when tape is peeled off the skin. Thus, the adhesive base material and octanoic acid and/or oleic acid are mixed with the adhesive base material/octanoic acid and/or oleic acid ratio by weight in the range from 100/1 to 100/10. Besides, octanoic acid and oleic acid can be used independently or together.

The said ingredient of lauric acid diethanolamide in this invention is also a chemical transdermal enhancer. If its addition is less than necessary, the enhancing effect on fentanyl transdermal delivery will be reduced; if its addition is larger than necessary, not only the adhesiveness of the adhesive base material will be reduced, but also the skin irritation will be increased. Thus, the adhesive base material and lauric acid diethanolamide are mixed with the adhesive base material/lauric acid diethanolamide ratio by weight in the range from 100/1 to 100/5.

The said ingredient of polyvinylpyrollidone (PVP) in this invention is used to increase both the solubility of fentanyl in adhesive base material and adhesion force of adhesive base material. A larger addition of PVP than necessary will reduce the adhesiveness of the adhesive base material which makes it difficult for the tape to adhere to the skin. Thus, the adhesive base material and PVP are mixed with the adhesive base material/PVP ratio by weight in the range from 100/5 to 100/20.

The said active ingredient of fentanyl is an extremely potent narcotic analgesic. A less addition of fentanyl than necessary will result in a lower transdermal permeation amount and a reduced analgesic effect. However, a larger addition than necessary will result in fentanyl crystal deposition which will also reduce the analgesic effect of fentanyl; on the other hand, it may result in breath inhibition because of over transdermal permeation of fentanyl due to the solubility increasing in adhesive base material. Thus, the adhesive base material and fentanyl are mixed with the adhesive base material/fentanyl ratio by weight in the range from 100/5 to 100/15.

The preparation method of transdermal administration type pharmaceutical preparation in tape form is not specifically limited. It could be prepared as follows:

To the adhesive base material which is the copolymer consisting of 70 to 80 weight % of 2-ethylhexyl acrylate (35 weight % of ethyl acetate solution) and 30 to 20 weight % of vinylpyrollidone, ethylene glycol monocetyl ether, octanoic acid and/or oleic acid, lauric acid diethanolamide, polyvinylpolypyrrolidone and fentanly was added in a concentration ratio of 100:1 to 10:1 to 10:1 to 5:5 to 20:5 to 15 by weight respectively, resulting in a coating solution. The coating solution is then applied to a release film, and dried, on the surface of which a backing is then laminated resulting in the desired transdermal administration type pharmaceutical preparation in tape form.

The thickness of adhesive base layer of the transdermal administration type pharmaceutical preparation in tape form according to this invention is not specifically limited. In general, the thickness is in a range of 50 to 5000 μm, preferably 100 to 2000 μm.

The transdermal administration type pharmaceutical preparation in tape form according to this invention has better cohesive force and appropriate skin adhesion, and could supply fentanyl gradually and at a constant rate over a prolonged period of application. Therefore, it is safe and pharmaceutically effective in use.

Examples of Adhesive Copolymerization

Adhesive A

A 500 ml flask was filled with 200 g ethyl acetate, 70 g 2-ethylhexyl acrylate, 20 g vinyl acetate, 10 g acrylic acid and 0.005 g azobisisobutyronitrile to form a polymerization solution. The solution was heated to 70 degree C. for 15 hours in a nitrogen atmosphere, resulting in adhesive A.

Adhesive B

A 500 ml flask was filled with 200 g ethyl acetate, 75 g 2-ethylhexyl acrylate, 25 g vinylpyrollidone and 0.005 g azobisisbutyronitrile to form a synthesize solution. The solution was heated to 70 degree C. for 15 hours in a nitrogen atmosphere, resulting in adhesive B.

Adhesive C

A 500 ml flask was filled with 200 g ethyl acetate, 95 g 2-ethylhexyl acrylate, 5 g acrylic acid and 0.005 g azobisisbutyronitrile to form a synthesize solution. The solution was heated to 70 degree C. for 15 hours in a nitrogen atmosphere, resulting in desired adhesive C.

### EXAMPLE 1 TO EXAMPLE 3

### Control 1 to Control 9

Given amounts of fentanyl, diethylene glycol monocetyl ether, oleic acid, lauric acid diethanolamide, and polyvinylpyrollidone were added to the given amount of adhesive (solid content) as shown in Table 1, thus resulting in a coating solution. The coating solution was then applied to a PET backing using a knife coater, and then dried at a temperature of 50 degree C. for 24 hours to form an adhesive base layer with a thickness of 200 μm, on the surface of which a release liner was then laminated resulting in the desired transdermal administration type pharmaceutical preparation in tape form.

TABLE 1

| | | | | Dithylene | Lauric | | |
|---|---|---|---|---|---|---|---|
| | | | | glycol | acid | | |
| | | | | monocetyl | diethanol- | | Polyvinyl- |
| | Adhesive | | Fentanyl | ether | amide | Oleic acid | pyrollidone |
| | No. | W* | W | W | W | W | W |
| Example 1 | A | 100 | 8 | 1.5 | 2.5 | 8 | 10 |
| Example 2 | B | 100 | 8 | 1.5 | 2.5 | 8 | 10 |
| Example 3 | D* | 100 | 8 | 1.5 | 2.5 | 8 | 10 |
| Control 1 | A | 100 | 8 | 0 | 2.5 | 8 | 10 |
| Control 2 | B | 100 | 8 | 1.5 | 0 | 8 | 10 |
| Control 3 | A | 100 | 8 | 1.5 | 2.5 | 0 | 10 |
| Control 4 | B | 100 | 8 | 1.5 | 2.5 | 8 | 0 |
| Control 5 | C | 100 | 8 | 1.5 | 2.5 | 8 | 10 |
| Control 6 | A | 100 | 8 | 1.5 | 2.5 | 8 | 10 |
| Control 7 | B | 100 | 8 | 1.5 | 10 | 8 | 10 |
| Control 8 | B | 100 | 8 | 1.5 | 2.5 | 15 | 10 |
| Control 9 | B | 100 | 8 | 1.5 | 2.5 | 8 | 30 |

*W: weight;
*D: DURO-TAC 87-2677

The fentanyl crystallization, skin irritation, adhesiveness and skin remain of the adhesive of the pharmaceutical preparation obtained above were examined, the results of which are shown in Table 2.

Fentanyl Crystallization Determination

The pharmaceutical preparation obtained above was maintained at room temperature for 72 hours, after which the growth of fentanyl crystals was observed by naked eyes.

Skin Irritation Test

The pharmaceutical preparation obtained above was applied to a shaved dorsal portion of the male rabbit, weighed 2.2 kg to 2.5 kg, with the patch size of 3.14 cm² in area (diameter of 2 cm in a cycle) for 24 hours. After removing the patch for 30 minutes, the irritation reaction was assessed using a 5-point scale of 0-4 as follows;

0=No erythema,

1=Very slight erythema,

2=Well defined erythema,

3=moderate erythema,

4=Strong erythema to eschar formation.

Skin Adherence Evaluation

Patch adherence was evaluated at the end of the application period. Just after the removal of patch, the skin irritation and residual adhesive on the skin were estimated.

TABLE 2

| | Fentanyl | Skin adherence, | |
|---|---|---|---|
| | crystal | and adhesive residual on skin | Skin irritation |
| Example 1 | X | Good, No resitual | 0 |
| Example 2 | X | Good, No resitual | 0 |
| Example 3 | X | Good, No resitual | 0 |
| Control 1 | X | Good, No resitual | 0.25 |
| Control 2 | X | Good, No resitual | 0.5 |
| Control 3 | ◯ | Good, No resitual | 0 |
| Control 4 | ◯ | Good, No resitual | 0.25 |
| Control 5 | ◯ | Good, No resitual | 0 |
| Control 6 | X | Weak cohesion, Residual | Not evaluated |
| | | adhesive | |
| Control 7 | X | Weak adherence, No residual | 2 |
| Control 8 | X | Weak cohesion, Residaul | Not evaluated |
| | | adhesive | |
| Control 9 | X | No skin adherence | Not evaluated |

X: indicates no fentanyl crystal or no adhesive remaining on skin;
◯: indicates fentanyl crystal appearance or adhesive remaining on skin

Skin Permeation Experiment

Example 1 to 3, and Control 1 to 5 were used to perform the skin permeation experiment with an improved Franze diffusion cell. The pharmaceutical preparation obtained above was mounted on the skin on the donor side. The receptor cell was filled with a binary vehicle of PEG 400/water=1:3 by weight. Cells were placed on a magnetic stirring plate with water bath maintained at 37° C. At the time intervals of 12, 24, 48 and 72 hours, 500 ul of receptor solution was taken from the receptor compartment and an equal volume of fresh receptor solution was replenished to maintain a constant volume. Samples were analyzed by HPLC method to calculate the accumulated permeation amount as indicated in Table 3.

TABLE 3
The cumulated permeation amount of fentanyl (n = 3)

Cumulated permeation amount of fentanyl (μg/cm²)

| | 12 hours | 24 hours | 48 hours | 72 hours |
|---|---|---|---|---|
| Example 1 | 43 | 98 | 188 | 235 |
| Example 2 | 43 | 101 | 227 | 277 |
| Example 3 | 51 | 111 | 256 | 295 |
| Control 1 | 23 | 41 | 91 | 119 |
| Control 2 | 24 | 45 | 99 | 123 |
| Control 3 | 16 | 30 | 43 | 49 |
| Control 4 | 13 | 23 | 31 | 46 |
| Control 5 | 16 | 21 | 35 | 45 |

1. A pharmaceutical preparation in tape form comprising
(1) a flexible backing, which is not permeable by ingredients,
(2) an adhesive base layer formed on said backing; said adhesive base layer consisting essentially of
(i) an adhesive base material of copolymer containing
(a) one or more (meth) alkyl acrylate monomers which has 4 to 10 carbon atoms in the alkyl group, and
(b) one or more monomers copolymerizable with the (a) monomer selected from the group of unsaturated ethylenically unsaturated monomers,
(ii) diethylene glycol monocetyl ether,
(iii) octanoic acid and/or oleic acid,
(iv) lauric acid diethanolamide,
(v) polyvinylpyrollidone;
(vi) active ingredient of fentanyl
wherein the concentration ratio of (i), (ii), (iii), (iv), (v), and (vi) are 100:1 to 10:1 to 10:1 to 5:5 to 20:5 to 15 by weight respectively.

2. A pharmaceutical preparation according to claim 1, wherein said adhesive base material is a copolymer containing said (a) monomer of 60 to 90% by weight selected from group of butyl acrylate, octyl acrylate, 2-ethylhexyl acrylate, 2-ethylhexyl methacrylate, and (b) monomer of 40 to 10% by weight of the total amount which is at least one selected from acrylic acid, methacrylic acid, and vinyl acetate.

3. A pharmaceutical preparation according to claim 1, wherein said adhesive base material is cross-linked copolymer.

4. A pharmaceutical preparation according to claim 1, wherein said adhesive base material is a copolymer containing monomer of 2-ethylhexyl acrylate of 70 to 80% by weight, and f vinylpyrollidone of 30 to 20% by weight.

5. A pharmaceutical preparation according to claim 2, wherein said adhesive base material is cross-linked copolymer.

6. A pharmaceutical preparation according to claim 2, wherein said adhesive base material is a copolymer containing monomer of 2-ethylhexyl acrylate of 70 to 80% by weight, and f vinylpyrollidone of 30 to 20% by weight.

7. A pharmaceutical preparation according to claim 3, wherein said adhesive base material is a copolymer containing monomer of 2-ethylhexyl acrylate of 70 to 80% by weight, and f vinylpyrollidone of 30 to 20% by weight.

8. A pharmaceutical preparation according to claim 5, wherein said adhesive base material is a copolymer containing monomer of 2-ethylhexyl acrylate of 70 to 80% by weight, and f vinylpyrollidone of 30 to 20% by weight.
